(19) United States
(12) Patent Application Publication
(10) Pub. No.: US 2011/0046455 A1
(43) Pub. Date: Feb. 24, 2011

(54) METHODS AND DEVICES FOR EXAMINING A PARTICULAR TISSUE VOLUME IN A BODY, AND A METHOD AND A DEVICE FOR SEGMENTING THE PARTICULAR TISSUE VOLUME

(76) Inventors: Arne HENGERER, Mohrendorf (DE); Andreas Kappel, Koenigstein (DE); Luis Lasalvia, White Plains, NY (US); Andrea Lichte, Marburg (DE); Thomas Mertelmeier, Erlangen (DE); Sven Meyburg, Erlangen (DE); Christoph Petry, Neuss (DE); Marcus Pfister, Bubenreuth (DE); Martin Requardt, Nurnberg (DE)

Correspondence Address: HARNESS, DICKEY & PIERCE, P.L.C., P.O.BOX 8910, RESTON, VA, 20195, US

(21) Appl. No.: 12/857,919

(22) Filed: Aug. 17, 2010

(30) Foreign Application Priority Data
Aug. 20, 2009 (DE) 10 2009 038 239.9

(51) Int. Cl. A61B 5/107; A61M 25/00; A61B 5/055; A61B 5/145; A61B 6/00; A61B 5/053; A61B 6/03

(52) U.S. Cl. 600/309; 600/587; 600/585; 600/420; 600/476; 600/434

(57) ABSTRACT

A tip of an elongate device is navigated into a particular tissue volume in order to examine the particular tissue volume in a body and part of the tissue volume is analyzed in real-time by way of a biosensor. In at least one embodiment, in the process, either the biosensor can be arranged on the tip of the device or the device includes a catheter, by which a substance to be analyzed is transported out of the tissue volume from the tip to the biosensor arranged at the proximal end of the catheter.

### PRIORITY STATEMENT

The present application hereby claims priority under 35 U.S.C. §119 on German patent application number DE 10 2009 038 239.9 filed Aug. 20, 2009, the entire contents of which are hereby incorporated herein by reference.

### FIELD

Embodiments of the present invention generally relate to methods and devices for examining a particular tissue volume in a human body in particular, and in at least one embodiment to a method and/or a device for segmenting this particular tissue volume.

### BACKGROUND

Localizing for example molecular biomarkers is necessary in various diagnostic methods in oncology, e.g. for treatment planning, for planning an intervention, for monitoring a therapy or for a check-up examination. For this, the prior art discloses the taking of samples from a tissue to be examined by means of a biopsy and subsequently letting these samples be analyzed by a person skilled in the art of histopathology. This procedure is an established method, although it is disadvantageous in that the examination of e.g. a patient is suspended because the histopathologic analysis requires a lot of time. The duration of the examination or a suspension of the examination to await the results from the histopathology is disadvantageous to a patient and can even have a negative influence on the result of the therapy. Moreover, specific infrastructure and a pathologist are required, which significantly increases the costs of the examination. In the prior art it is therefore often conventional for these biomarkers to be determined merely in a systemic fashion, i.e. without spatial resolution, by way of blood samples.

### SUMMARY

At least one embodiment of the present invention is directed to localization of biomarkers and hence the examination of a particular tissue to be accelerated.

According to at least one embodiment of the invention, a device is disclosed for examining a particular tissue volume, an analysis apparatus is disclosed for examining a particular tissue volume, a segmentation device is disclosed for segmenting a particular volume, a method is disclosed for segmenting a particular volume and/or a method is disclosed for examining a particular tissue volume. The dependent claims define preferred and advantageous embodiments of the present invention.

Within the scope of at least one embodiment of the present invention, a device is provided for examining a particular tissue volume in a human body in particular. Here the device comprises a guiding device and a biosensor, which is attached to the guiding device at a suitable location. The device is developed such that the biosensor can be guided through the body to part of the tissue volume to be examined with the aid of the guiding device and that the biosensor is designed for real-time analysis of this part of the tissue volume as a result of the biosensor for example analyzing bodily fluids, such as interstitial fluids, and tissue. In the process, the biosensor is in particular attached to a distal end of the guiding device.

On the one hand, the biosensor can as it were form the distal end of the device in order to thus be guided to the particular tissue volume by means of the guiding device. However, it can also be advantageous for the biosensor to be arranged slightly offset (spaced apart) from the distal end of the guiding device so that cell fragments, which are loosened as a result of the guiding device moving to the particular tissue volume, do not obstruct or even damage the biosensor.

In the process, the biosensor can first of all be arranged on the outside of the guiding device. Moreover, the biosensor can be arranged within the guiding device such that the biosensor is as it were protected by the guiding device. If the biosensor is arranged offset from the tip or the distal end of the device, a distance from the tip of the device of between 1 cm and 5 cm was found to be advantageous.

Here the guiding device is any device that guides the biosensor to the tissue volume to be examined. Here the guiding device can be a biopsy needle, a laparoscopic device for minimally invasive surgery, a probing instrument, a guide wire or else a catheter, as will still be explained below.

Within the scope of at least one embodiment of the present invention, the real-time analysis is in this case understood to be an analysis of a tissue part carried out with the aid of the biosensor, without this using a device outside of the body for processing the sample, as is conventional in pathological methods according to the prior art. Here a biosensor is understood to be a sensor equipped with biological components. In this case, a biosensor is based on a biological identification element (biochemical receptor) and a signal converter (magnetic amplifier), which are in direct spatial contact, and an electronic amplifier. In order to identify the substances to be determined, a biosensor utilizes biological systems such as antibodies, enzymes, organelles or microorganisms, oligonucleotides, aptamers, peptides, receptors, or sugar structures.

As a result of the biosensor carrying out the examination of the particular tissue volume and coming into direct contact with the tissue volume to be analyzed or the bodily fluid to be analyzed, the tissue volume can be analyzed very rapidly compared to an examination according to the prior art based on a biopsy.

In another embodiment according to at least one embodiment of the invention, the guiding device comprises a catheter or the guiding device is a catheter. In this embodiment, the device moreover comprises a pump and a container. The pump and container are connected to the catheter at a proximal end of the catheter such that the pump can pump a substance (e.g. a liquid) situated in the container through the catheter to the distal end of the catheter. As already indicated previously, the biosensor can be arranged at the distal end of the catheter. However, it is also possible for the biosensor to be arranged offset from the tip (of the distal end) of the catheter. For this, e.g. a lateral opening in the catheter can be connected to the biosensor, and so part of the tissue volume to be examined can be supplied to the biosensor via the catheter and said opening. However, it is also feasible for the biosensor to be arranged within the catheter, wherein part of the tissue volume to be examined can also be supplied to the biosensor via the catheter.

Here the distal end is understood to be an end that is guided to the particular tissue volume or in direct contact with the particular tissue volume, while the proximal end is the other end of a two-ended device (of e.g. the device according to at least one embodiment of the invention, the catheter, the guiding device).

Such a device according to at least one embodiment of the invention can firstly transport a substance to the part of the particular tissue volume to be examined, by means of which substance the biosensor carries out its real-time analysis. Moreover, a substance may also be supplied to the particular tissue volume depending on the real-time analysis by the biosensor in order to initiate therapy with the aid of this substance as a function of the results determined by the biosensor. A combination of these two variants is also possible, and so the same device according to the invention can supply a substance for the real-time analysis by means of the biosensor and a substance for the therapy. This can also be the same substance in this case.

A radiofrequency (RF) coil can be attached to the biosensor in order to improve imaging and localization of the biosensor by way of a magnetic resonance scanner. For this, the magnetic resonance scanner emits particular pulse sequences that are matched to the coil or coils attached to the biosensor in order to localize the biosensor as precisely as possible in real time.

Localizing the biosensor, for example in an image of that body section in which the biosensor is guided to the tissue volume to be examined, assists an operator in guiding the biosensor as precisely as possible to the part of the tissue volume determined in advance.

The results from the biosensor can be transmitted to the outside via a cable (electrical conductor or optical waveguide) within the guiding device or via a wireless connection (e.g. Bluetooth).

A further device for examining a particular tissue volume in a body is also provided within the scope of at least one embodiment of the present invention. This further device according to at least one embodiment of the invention comprises a sample acquisition device, a catheter and a biosensor. Here the sample acquisition device is connected to the distal end of the catheter and the biosensor is connected to the proximal end of the catheter such that a sample can be transported through the catheter from the sample acquisition device to the biosensor. The device is developed such that the sample acquisition device can be guided through the body into a predetermined part of the tissue volume and that the biosensor is designed for real-time analysis of the sample.

In this device according to at least one embodiment of the invention, the biosensor can be arranged beyond the five-Gauss line such that the biosensor can also have ferromagnetic materials, which would have a negative influence on the imaging and hence the guiding of the device according to at least one embodiment of the invention with the aid of this imaging when used in a magnetic resonance scanner (for example, a measured value transducer of the biosensor could be made up of ferromagnetic materials). Moreover, if the biosensor is situated beyond the five-Gauss line, it is not subjected to a negative influence from the magnetic field of the magnetic resonance scanner when used in a magnetic resonance scanner. Here the five-Gauss line is understood to be a line on which the magnetic field has a strength of five Gauss, and so the magnetic field in a region beyond the five-Gauss line has a strength of less than five Gauss. In order to avoid the negative effects of the magnetic field of the magnetic resonance scanner, the biosensor can also be arranged within the five-Gauss line if the biosensor has appropriate magnetic shielding.

The further device according to at least one embodiment of the invention can also additionally comprise a pump and a container. Here the pump and the container together with the biosensor are arranged at the proximal end of the catheter such that the pump can pump a substance situated in the container through the catheter to the distal end of the catheter.

By way of example, this affords the possibility of supplying a particular substance, for example nanoparticles, to the tissue volume to be examined and of subsequently transporting part of this substance, which has in particular reacted with the tissue volume to be examined, via the catheter to the biosensor by means of the sample acquisition device. Analysis of this substance transported to the biosensor by means of the biosensor affords an analysis of the predetermined tissue volume.

A further option consists of supplying a particular substance to this tissue volume in the direct vicinity of the pathology through the catheter in order to thereby initiate a therapy, for example combating a tumor, if a particular analysis of the particular tissue volume by way of the biosensor is present.

The sample acquisition device can comprise an RF coil for improved localization during imaging by way of a magnetic resonance scanner.

While the sample acquisition device can also sever part of the tissue to be examined, with this part then being transported to the biosensor via the catheter, the sample acquisition device can also (merely) be an applicator, by which the substance available in the container is applied into the tissue part. In this case, a substantially liquid sample is suctioned off via the catheter (e.g. with the aid of the pump) in order to then be examined by the biosensor.

The following embodiments according to the invention relate to embodiments of both devices according to the invention described above.

According to a first of these embodiments according to the invention, the container is developed to hold particles (i.e. beads, microbeads or the like (diameter in the μm range) or nanoparticles (diameter in the nm range)) with capture antibodies (or other molecules with a high affinity to a biomolecule to be detected) or (antisense-) oligonucleotides. Here the device is designed such that the particles can be pumped through the catheter to the distal end of the catheter (and hence into the tissue volume to be examined) by means of the pump and that the biosensor can carry out real-time analysis with the aid of the particles, which have at least in part again been transported back to the proximal end of the catheter from the tissue volume to be examined.

In the process, the capture antibodies or (antisense-) oligonucleotides attach to the analytes (more particularly the biomarkers) within the tissue to be examined and so these analytes are transported to the biosensor together with the capture antibodies or (antisense-) oligonucleotides for analysis.

The biosensor can be based on at least one of the following techniques or methods:
The quartz crystal microbalance (QCM) technique
The change in frequency of a quartz crystal resonator is measured by this technique. This technique allows weighing of very small masses down to the nanogram range, determining the thickness of thin slices into the sub-nanometer range and offers many sensory options such as determining the viscosity, the degree of swelling, the gas concentration, the density and the humidity. Moreover, it is possible to examine rheological properties in the high-frequency range (>10⁶ Hz).
Surface plasmon resonance: A biochemical interaction—for example of an antibody with a protein—with unmarked molecules can be detected with the aid of surface plasmon resonance (SPR). This technology is based on the fact that polarized light undergoes total reflection when incident on a thin layer of gold and simultaneously triggers a plasmon resonance in the gold layer. A shadow in the reflected light resulting therefrom changes proportionally with the mass bound to the chip surface, as a result of which very small changes in mass can be detected. The change in the bound mass is expressed in RU, wherein 1 RU corresponds to the mass of 1 pg/mm².
Calorimetry: Exothermal chemical reactions lead to a release of heat. The increase in temperature resulting therefrom depends on the amount of substance of the chemical reaction partners. The temperature is acquired using miniaturized semiconductor thermistors.
Electrochemical detection (amperometry or potentiometry): In the case of amperometry, the current flow is measured at constant voltage with the aid of electrodes. It is suitable for metabolic products that can easily be oxidized or reduced. Potentiometry is used in the case of ionic reaction products (e.g. NH₄⁺; CO₃²⁻; H⁺). These ions are determined quantitatively on the basis of their electrochemical potential at a measurement electrode. This also affords the possibility of determining changes in the pH-value within a tissue volume.
Detecting evanescent fields or waves in respect of the tissue to be examined.
Measuring the electrical impedance of the tissue to be examined.
Carrying out a fluorescence-optical examination, such as e.g. fluorescence spectroscopy, of the tissue to be examined.
Carrying out a spectrophotometric analysis, i.e. determining the extinction or transmission of electromagnetic radiation within the tissue and the surrounding liquid.
Carrying out an enzymatic analysis, i.e. an analysis of the enzymes, of the tissue to be examined, or the detection of enzyme substrates by coupled enzymatic reactions, with the enzymes being part of the biosensor.

The biosensor can be based on any measured value transformation technology that can be miniaturized and automated.

In a further embodiment relating to both devices according to at least one embodiment of the invention, the biosensor is made up of an arrangement of a plurality of individual biosensors, with each of these biosensors analyzing one or more particular parameters of the tissue volume to be examined.

The region of the device according to at least one embodiment of the invention, in which—depending on the implementation—the biosensor or the sample acquisition device is situated, or the guiding device or the catheter can consist of a material that causes a slight disturbance in MRI images. The tip or the guiding device or catheter can, as a further option, be coated by a material that reduces the relaxation constant of the surrounding tissue. As a result, the tip or the guiding device or the catheter appears with contrast in an MRI image, which is also referred to as passive visualization.

The container can comprise a plurality of sub-containers (e.g. in the form of a rotating system) in order to be able to emit different substances.

The emission of particles or a different substance by the device according to at least one embodiment of the invention and the subsequent distribution of the substance in the tissue can be monitored by imaging methods.

Moreover, an analysis apparatus for examining a particular tissue volume in a body is provided within the scope of at least one embodiment of the present invention. Here the analysis apparatus comprises a robotic arm, a controller and a device according to at least one embodiment of the invention, which is described above. The analysis apparatus is developed such that firstly images of the body, which are generated, for example, by a magnetic resonance scanner or by another imaging device, and a position within the particular tissue volume, which is determined, for example, by a medical practitioner by means of the above-described images, can be predetermined for the controller. Depending on whether the biosensor or the sample acquisition device is arranged at the distal end of the device according to at least one embodiment of the invention, the biosensor or the sample acquisition device of the device according to at least one embodiment of the invention is automatically navigated in the body by the robotic arm. Here the controller controls the robotic arm as a function of the images and the predetermined position such that the biosensor or the sample acquisition device is automatically (without manual operation) guided to the predetermined position.

This analysis apparatus according to at least one embodiment of the invention increases the degree of automation of an analysis of a particular tissue volume in a for example human body, as a result of which human errors, such as guiding the biosensor or the sample acquisition device to a wrong position within the tissue volume to be examined, are avoided.

Moreover, a segmentation device for segmenting a particular volume in a body is provided within the scope of at least one embodiment of the present invention. Here the segmentation device comprises a computational unit and a device according to at least one embodiment of the invention, as is described above. The segmentation device is developed such that images of the particular volume, which are generated, for example, by a magnetic resonance scanner or by another imaging device, and the analysis results from the biosensor of the device according to at least one embodiment of the invention can be supplied to the computational unit. The computational unit generates a segmentation of the particular volume as a function of these images and the analysis results from the biosensor. Here a segmentation is understood to be the generation of content-connected regions (e.g. organs, tissue types, pathologies, gene expression regions, cells) by combining neighboring pixels or voxels in accordance with a particular homogeneity criterion in order to for example display these regions with appropriate coloring.

A method for segmenting a particular volume in a body is also provided within the scope of at least one embodiment of the present invention. Here images of the particular volume and results from a biosensor are prescribed and the segmentation of the particular volume is carried out as a function of these images and the results from the biosensor.

By considering the results from the analysis from the biosensor in the segmentation, the segmentation, for example the distinction between, or the delineation of, various organs (or tissue types, pathologies, gene expression regions, cells), can be carried out more accurately than would be the case without considering these results.

By way of example, a volume of a pathology, determined as a function of a segmentation determined exclusively on the basis of images (that is to say without the results from the biosensor), can be verified by a substance concentration, e.g. a biomarker concentration or a concentration of a therapeutic agent supplied in advance, either in the surroundings of the pathology or in the pathology itself. In the case of discrepancies, i.e. the segmentation results not corresponding to the measured substance concentrations, the segmentation can be modified appropriately, i.e. matched to the results from the biosensor.

Finally, a method for examining a particular tissue volume in a body is provided within the scope of at least one embodiment of the present invention. Here an elongate device is navigated into the particular tissue volume and part of the tissue volume is analyzed in real time by way of a biosensor, i.e. the biosensor carries out real-time analysis.

In an embodiment according to the invention, the biosensor is in this case arranged at the tip of the elongate device such that said biosensor is guided directly to the part of the tissue volume to be examined.

Here the tip of the elongate device can be navigated or guided under direct imaging of a region of the body. Here this region of the body corresponds to the particular tissue volume and a section within the body in which the tip is guided to the particular tissue volume.

In a further embodiment according to the invention, the biosensor is arranged spatially separate from the tip of the probing instrument. The advantage offered by this method is that tissue or cell fragments, which may be generated during the positioning of the guiding device in the tissue, do not come into direct contact with the sensor surface and this prevents unspecific occupation of the sensor with cells, cell fragments, tissue or tissue fragments.

Here the direct imaging can be undertaken by way of:
fluoroscopy,
magnetic resonance imaging,
positron emission tomography,
computed tomography, or
by way of a three-dimensional data record generated from the corresponding region of the body with the aid of magnetic resonance imaging, computed tomography or computed tomography angiography, and superposed on a two-dimensional fluoroscopy image of this region.

In the case of angiographic systems, three-dimensional image data records (generated by computed tomography, magnetic resonance imaging or computed tomography angiography) illustrating anatomical details (e.g. of the tissue to be examined) generated even before using the device according to the invention, or generated while the latter is used, can be registered and superposed on a two-dimensional X-ray fluoroscopy image in order to improve the navigation of the device according to at least one embodiment of the invention.

For the purpose of navigating the tip of the device according to at least one embodiment of the invention, it is advantageous for the tip to be imaged in three dimensions by means of an electromagnetic method, an optical method or an X-ray method operating from different directions and to be registered in a three-dimensional data record. This three-dimensional data record is combined with a registered three-dimensional data record of the region of the body in which the tip is guided. The tip can advantageously be navigated within the body by way of these combined three-dimensional data records.

Here a registered data record is understood to be a data record consisting of a plurality of images of the same body section. Here a transformation is known for each of these images in order to bring said image into best possible agreement with every other image in the data record such that particular mutually corresponding prominent regions of the images are congruent. Here the images (to be registered) of the data record differ from one another because they were recorded from different positions and/or with different imaging devices.

According to at least one embodiment of the invention, it is possible for the results from an analysis carried out by the biosensor to be transferred to an (e.g. three-dimensional) data record generated by direct imaging and to be combined with the latter data record in order to thereby improve the imaging and an analysis of the predetermined tissue volume.

Combining the results like this allows for a more detailed diagnosis than if the diagnosis is based exclusively on the imaging or exclusively on the results from the biosensor.

More particularly, if the method according to at least one embodiment of the invention is carried out on an angiography system, results from the analysis carried out by the biosensor can be combined with a two-dimensional fluoroscopy image of the tissue volume in order to thereby improve the imaging and an analysis of the particular tissue volume.

Moreover, it is also possible for results from an analysis carried out by the biosensor to be combined with results from a segmentation carried out dependent on the imaging in order to improve the results from the segmentation.

By way of example, the analysis results from the biosensor can be combined with a registered three-dimensional data record of the corresponding tissue section, with these results from the biosensor correlating with information obtained within the scope of a pre-process of the three-dimensional data record (e.g. in the form of an automatic, manual or semi-automatic segmentation). In the process, a particular concentration of a biomarker or a particular concentration of a therapeutic agent previously supplied to the tissue section to be examined (wherein this concentration has been measured by the biosensor or by another biosensor) for example correlates with that organ size or section size of the tissue section to be examined that was estimated by way of the segmentation results.

In an embodiment according to the invention, a plate provided with a grid structure is arranged in the vicinity of the tissue volume to be examined. The part of the tissue volume that should be analyzed by way of the biosensor is localized as a function of the grid structure with the aid of the direct imaging.

This affords the possibility of the tip of the device according to at least one embodiment of the invention being automatically guided to the predetermined part of the tissue volume by way of a robotic arm as a function of images generated by a magnetic resonance imaging scanner.

By way of example, in mammography, a section of the breast can be arranged between two compression plates, with one of these plates having a grid structure. The position of a tumor within the breast section is localized by way of contrast-enhancing imaging from a magnetic resonance imaging scanner. In the process, the position of the tumor or the lesion is determined in respect of the grid structure and a position for inserting a needle (this corresponds to the catheter of the device according to the invention) and an insertion depth are calculated as a function thereof. After inserting the needle to the predetermined position, the position of the needle is once again verified by means of up-to-date MRI images. If it is deemed necessary, the position of the needle is corrected before a sample is taken, transported to the biosensor and analyzed. Here the needle can be guided to the predetermined position by means of a robotic arm operating on piezoelectric or pneumatic principles and arranged within the magnetic resonance scanner next to the patient. Here the robotic arm is controlled automatically as a function of MRI images. A magnetic resonance imaging scanner with an internal diameter of more than 60 cm is advantageous when using a robotic arm so that the movement range of the robotic arm need not be restricted where possible.

In one embodiment of the method according to the invention, ferrous particles are supplied to the tissue volume by means of a catheter. Depending on the analysis of the tissue volume by the biosensor, the tissue volume is subsequently excited by an alternating magnetic field in order to increase the temperature in the tissue volume. In addition to the option of increasing the temperature in the tissue volume by way of an alternating magnetic field, the ferrous particles can also be displayed well by imaging methods operating using a magnetic resonance imaging scanner.

In this embodiment, an active-agent solution or a magnetic fluid, which contains iron oxide particles, such as very small iron oxide particles (VSOP), ultra small iron oxide particles (USPIO), or iron oxide particles that can for example also be coated by a chemotherapeutic agent (e.g. doxorubicin), can be injected into the tissue volume. For this, the solution is guided to the tissue volume from a container by way of a pump and a guide system or a catheter. The alternating magnetic field, which is generated by the magnetic resonance scanner and has a frequency of e.g. 100 kHz and a variable field strength of e.g. 0-18 kA/m, heats the tissue volume to 2-10° C. above body temperature due to the movement of the magnetic (ferrous) particles, as a result of which the tumor tissue is destroyed. This heating by way of magnetic particles is also known as local hyperthermia therapy.

Reference is made to the fact that the good display properties of the ferrous particles in MRI and/or the therapy option of supplying particular medical active agents to the predetermined tissue volume in a targeted fashion by way of the ferrous particles are independent of a hyperthermia therapy. That is to say these display properties and/or this therapy option are/is also available without hyperthermia therapy.

In an alternative embodiment according to at least one embodiment of the invention for local hyperthermia therapy, an ultrasound probe or a radiofrequency ablation device (device for ablating material by heating) can be present in the catheter.

In a further embodiment of the method according to at least one embodiment of the invention, particles containing a medical substance (e.g. a chemotherapeutic agent) encapsulated in a thermo-sensitive substance are supplied to the predetermined tissue volume by way of a catheter. Depending on the analysis of the tissue volume carried out by the biosensor, the tissue volume is heated in a targeted fashion, for example by way of local hyperthermia, in order to release the medical substance in the predetermined tissue volume only where possible. For this, the active agent is for example encapsulated in a heat-sensitive polymer that melts in the case of a small increase in temperature and thus releases the active agent.

At least one embodiment of the present invention offers at least one of the following advantages:
The analysis generates results from an undiluted sample. The biomarkers (e.g. proteins, peptides, and nucleic acids), chemical analytes (e.g. ions) or physical markers (e.g. temperature, electromagnetic radiation, etc.) are acquired (and also analyzed) at the site of the tissue volume to be examined instead of being measured in a systemically diluted fashion within the entire (blood) volume of the patient.
A real-time measurement of dynamic processes within the body is possible.
A repeated or continuous measurement of the analytes is possible provided the sensor remains in the tissue for a relatively long time or is implanted in a permanent fashion.
The combined acquisition of biomarkers and carrying out of therapy steps leads to an improved workflow.
Analytes are localized by a combination of imaging methods and analysis results from the biosensor.

At least one embodiment of the present invention is particularly suitable for analyzing a tissue situated in a body. However, the present invention is not limited to this preferred field of application but can also be used for carrying out therapy steps. At least one embodiment of the present invention can moreover also be used for controlling or monitoring a therapy by using the biosensor to determine concentrations of biomarkers for necrosis, such as intracellular proteins (TNF-α) or RNA.

At least one embodiment of the present invention can be used for examining the (female) breast, the liver, the prostrate, the brain, the spinal cord and/or the heart. When used for a brain examination (biopsy), at least one embodiment of the present invention provides an option for completely removing lesions whilst at the same time leaving healthy brain tissue almost entirely unharmed as a result of the real-time tissue analysis ability.

### BRIEF DESCRIPTION OF THE DRAWINGS

Hereinbelow, the present invention is described in detail with reference to the figures with the aid of example embodiments according to the invention.

FIG. 1 schematically illustrates a first embodiment according to the invention of a device for examining a particular tissue volume in a body.

FIG. 2 schematically illustrates a second embodiment according to the invention of a device for examining a particular tissue volume in a body.

FIG. 3 schematically illustrates an analysis apparatus according to an embodiment of the invention together with a magnetic resonance scanner.

FIG. 4 schematically illustrates a segmentation device according to an embodiment of the invention together with a magnetic resonance scanner.

FIG. 5 illustrates a flowchart of a method according to an embodiment of the invention for examining a particular tissue volume in a body.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

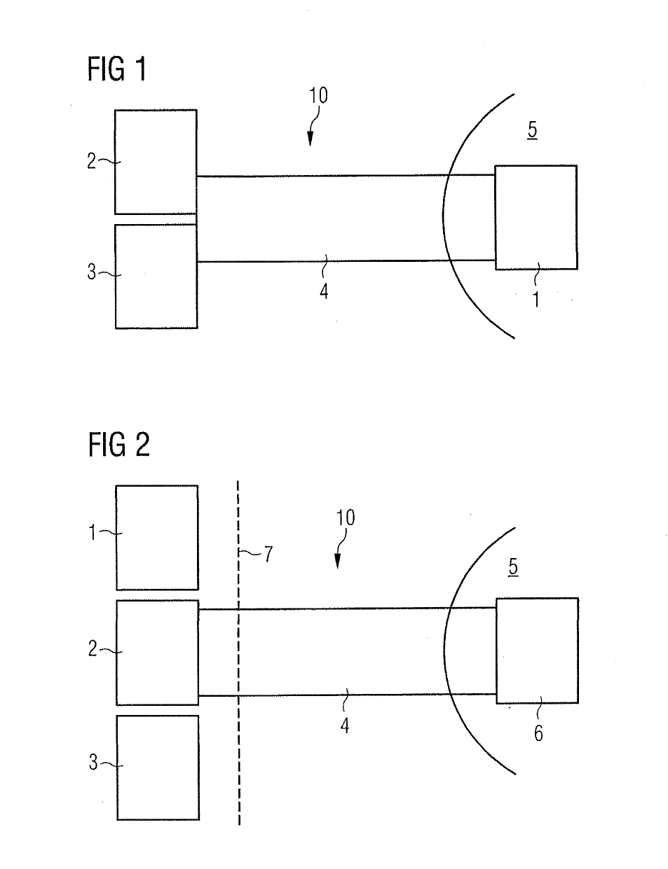

FIG. 1 illustrates a device 10 according to an embodiment of the invention for analyzing a particular tissue volume in a body. The elongate device 10 according to an embodiment of the invention comprises a biosensor 1, a pump 2, a storage container 3 and a catheter 4. Here the biosensor 1 is attached to the distal end of the catheter 4, whereas the pump 2 and the container 3 are attached to the proximal end of the catheter 4. The device 10 is developed such that the biosensor 1 at the tip of the catheter 4 can be inserted into a tissue volume to be examined in a human body 5.

Once the biosensor 1 has been inserted into the tissue to be examined, it carries out an examination there in real time by for example determining a concentration of particular biomarkers without requiring an apparatus arranged outside of the body 5 for this. A substance, for example a solution with an active agent, situated in the container 3 can be supplied to the tissue via the catheter 4 with the aid of the pump 2. By way of example, this affords the possibility of, in one work flow, firstly carrying out an examination of the tissue (by means of the biosensor 1) and secondly carrying out a treatment depending on the examination results by transporting an active agent situated in the container 3 to the tissue in a targeted fashion via the catheter 4 with the aid of the pump 2.

FIG. 2 illustrates a further embodiment of the device 10 according to an embodiment of the invention. In this embodiment, the biosensor 1 is arranged at the proximal end of the catheter 4, whereas a sample acquisition device 6 is attached to the distal end of the catheter 4. Once the sample acquisition device 6 has been guided into the tissue to be examined by means of the catheter 4, samples can be gathered by the sample acquisition device 6 and can be transported to the biosensor 1 via the catheter 4, for example by way of the pump 2.

Compared to the embodiment illustrated in FIG. 1, the embodiment illustrated in FIG. 2 is advantageous in that the biosensor 1 in FIG. 2 can also comprise ferromagnetic materials and can also utilize measurement methods that would be disturbed by an overly strong magnetic field, which is particularly advantageous when using the device 10 according to an embodiment of the invention in a magnetic resonance scanner. It is for this reason that the biosensor 1 is arranged beyond the five-Gauss line 7 in the device 10 illustrated in FIG. 2, which means that the biosensor 1 is situated in a region in which the magnetic field strength is less than five Gauss. It would also be possible for the biosensor 1 to be arranged within the five-Gauss line 7 if the biosensor has appropriate magnetic shielding.

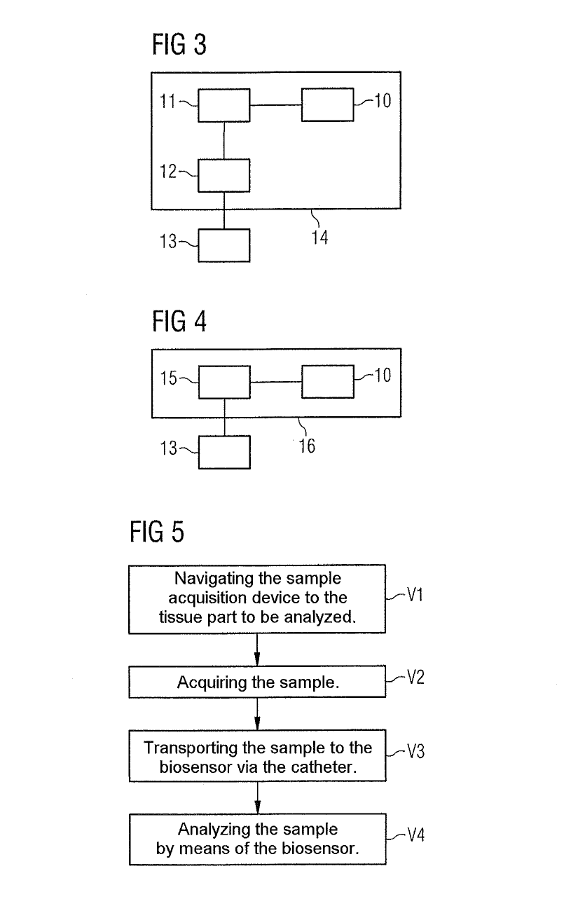

FIG. 3 illustrates an analysis apparatus 14 according to an embodiment of the invention, which comprises a device 10 according to an embodiment of the invention for examining a particular tissue volume in a body 5, a controller 12 and a robotic arm 11. The analysis apparatus 14 according to an embodiment of the invention is developed such that the device 10 according to the invention is automatically guided to a predetermined point within the tissue volume to be examined by the robotic arm 11, with the robotic arm 11 being controlled by the controller 12 in the process. In other words, the biosensor 1, if this is an embodiment of the device 10 in which the biosensor 1 is attached to the tip of the catheter 4 or a guiding device of the device 10, or the sample acquisition device 6, if this is an embodiment in which the sample acquisition device 6 is attached to the tip of the catheter 4 of the device 10, is navigated directly to the predetermined point within the tissue by the robotic arm 11.

The controller 12 obtains images of the body section in which the device 10 is guided to the tissue volume to be examined as information in order to control the robotic arm 11 and hence the distal end of the device 10 with the aid of these images. The images of the body section can be generated by any imaging method, such as a method operating using a magnetic resonance scanner 13, as illustrated in FIG. 3.

FIG. 4 illustrates a segmentation device 16 according to an embodiment of the invention, which comprises a device 10 according to the invention in addition to a computational unit 15. The computational unit 15 is supplied with images of a body section in which a particular tissue volume is also analyzed by way of the biosensor 1 of the device according to an embodiment of the invention. In order to be able to display particular organs or regions appropriately to for example a medical practitioner, the computational unit carries out a segmentation using these images, which are generated by any imaging method (in the illustrated case by a magnetic resonance scanner 13). In the process, the segmentation method implemented in the computational unit 15 also evaluates results from the biosensor in order to thereby improve or correct results from the segmentation method.

Here it should be noted that the results from the segmentation method are improved by the analysis results from the biosensor 1 and also the diagnosis results from the biosensor 1 are improved by the results from the segmentation method. That is to say the two methods complement each other in a synergistic fashion. The imaging or segmentation supplies the anatomical context while the biosensor 1 in particular contributes molecular-biological information, such as information relating to the antigen status, information relating to the gene expression, etc.

FIG. 5 illustrates an embodiment of a method according to the invention for examining a particular tissue volume in a body 5.

In the process, the sample acquisition device 6 is guided or navigated to the tissue parts to be analyzed in a first method step V1. The sample acquisition device 6 is used in a second method step V2 to gather a sample from the tissue volume to be analyzed and to transport the sample to the biosensor 1 via the catheter 4 in a third method step V3. Thereupon the biosensor 1 analyzes this sample in real time in a fourth method step V4 and transmits the results thereof to an apparatus (not illustrated), which displays these results for example to a medical practitioner in an appropriate fashion.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A device for examining a tissue volume in a body, comprising:
a guiding device; and
a biosensor disposed on the guiding device, wherein the biosensor is guidable through the body into part of the tissue volume via the guiding device and wherein the biosensor is designed for real-time analysis of the part of the tissue volume.

2. The device as claimed in claim 1, wherein the guiding device comprises a catheter, a pump and a container, the catheter connecting a distal region, on which the biosensor is arranged, to a proximal region at a distance therefrom, and wherein the pump and container are connected to the catheter in the proximal region such that the pump can pump a substance situated in the container through the catheter to the distal region.

3. The device as claimed in claim 1, wherein a coil is arranged on the biosensor to improve imaging of the biosensor by way of a magnetic resonance scanner.

4. A device for examining a tissue volume in a body, comprising:
a sample acquisition device;
a catheter; and
a biosensor, wherein the catheter connects the sample acquisition device to the biosensor such that a sample is transportable through the catheter from the sample acquisition device to the biosensor, wherein the sample acquisition device is guidable through the body into part of the tissue volume and wherein the biosensor is designed for real-time analysis of the sample.

5. The device as claimed in claim 4, further comprising a pump and a container, the pump and the container being connected to the catheter at a proximal end of the catheter on which the biosensor is arranged such that the pump can pump a substance situated in the container through the catheter to a distal end of the catheter.

6. The device as claimed in claim 4, wherein a coil is arranged on the sample acquisition device to improve imaging of the sample acquisition device by way of a magnetic resonance scanner.

7. The device as claimed in claim 2, the container is developed to hold particles with antibodies, oligonucleotides, enzymes or other specific binding molecules, wherein the device is developed such that the pump can be used to pump the particles through the catheter to the distal end of the catheter and wherein the biosensor is developed for real-time analysis by way of the particles.

8. The device as claimed in claim 1, wherein the biosensor comprises at least one of the following devices:
a device for applying the quartz crystal microbalance technique,
a detector for evanescent fields,
an impedance sensor,
a device for applying surface plasmon resonance technology,
a device for applying calorimetry,
a device for carrying out electrochemical detection,
a device for carrying out fluorescence-optical analysis,
a device for carrying out spectrophotometric analysis, and
a device for carrying out enzymatic analysis.

9. The device as claimed in claim 1, wherein the biosensor comprises an arrangement of a plurality of biosensors in order to analyze a plurality of parameters of the part of the tissue volume.

10. An analysis apparatus for examining a tissue volume in a body, comprising:
a robotic arm,
a controller, and
a device as claimed in claim 1, wherein the analysis apparatus is developed such that images of the body and a position within the tissue volume can be predetermined for the controller, wherein the robotic arm is useable to navigate the biosensor or the sample acquisition device of the device in the body, and wherein the controller controls the robotic arm as a function of the images and the position such that the biosensor or the sample acquisition device is guided to the position.

11. A segmentation device for segmenting a volume in a body, comprising:
a computational unit; and
a device as claimed in claim 1, wherein the segmentation device is developed such that images of the volume and analysis results from the biosensor of the device are suppliable to the computational unit, wherein the computational unit us useable to generate a segmentation of the volume as a function of the images and the analysis results.

12. A method for segmenting a volume in a body, wherein images of the volume and results from a biosensor are prescribed, comprising:
carrying out the segmentation of the volume as a function of the images and the results from the biosensor.

13. The method as claimed in claim 12, wherein an organ size determined as a result from a segmentation carried out solely as a function of the images is adapted as a function of a substance concentration determined by way of the biosensor.

14. A method for examining a tissue volume in a body, comprising:
navigating a tip of an elongate device into the tissue volume; and
analyzing part of the tissue volume in real time via a biosensor.

15. The method as claimed in claim 14, wherein the biosensor is arranged at the tip of the elongate device.

16. The method as claimed in claim 14, wherein the biosensor is arranged offset from the tip of the elongate device on the elongate device.

17. The method as claimed in claim 14, wherein the tip is navigated under direct imaging of a region of the body comprising the tissue volume and a region in which the tip is moved to the tissue volume.

18. The method as claimed in claim 17, wherein the direct imaging is at least undertaken via fluoroscopy or via magnetic resonance imaging or via positron emission tomography or via computed tomography or via a three-dimensional data record generated from the region of the body with the aid of magnetic resonance imaging, computed tomography or computed tomography angiography, and superposed on a two-dimensional fluoroscopy image.

19. The method as claimed in claim 17, wherein the tip of the elongate device is imaged in three dimensions by way of an electromagnetic method, an optical method or an X-ray method operating from different directions and registered in a three-dimensional data record, wherein the three-dimensional data record is combined with a registered three-dimensional data record of the region of the body, and wherein the tip is navigated with the aid of the combined three-dimensional data records.

20. The method as claimed in claim 17, wherein results from an analysis carried out by the biosensor are transferred to a data record generated by direct imaging and combined with the latter to thereby improve the imaging and an analysis of the particular tissue volume.

21. The method as claimed in claim 17, wherein results from an analysis carried out by the biosensor are combined with a two-dimensional fluoroscopy image of the tissue volume to thereby improve the imaging and an analysis of the particular tissue volume.

22. The method as claimed in claim 17, wherein results from an analysis carried out by the biosensor are combined with results from a segmentation carried out by way of the imaging to improve the results from the segmentation.

23. The method as claimed in claim 17, wherein a plate with a grid structure is arranged in the vicinity of the tissue volume and the part of the tissue volume is localized as a function of the grid structure with the aid of the direct imaging.

24. The method as claimed in claim 23, wherein the tip of the elongate device is automatically navigated by way of a robotic arm on the basis of images generated by a magnetic resonance imaging scanner.

25. The method as claimed in claim 17, wherein the direct imaging is undertaken by use of magnetic resonance imaging, and wherein a coil is arranged on the tip of the elongate device to improve imaging of the tip.

26. The method as claimed in claim 14, wherein the biosensor comprises at least one of the following devices:
a device for applying the quartz crystal microbalance technique,
a detector for evanescent fields,
an impedance sensor,
a device for applying surface plasmon resonance technology,
a device for applying calorimetry,
a device for carrying out electrochemical detection,
a device for carrying out fluorescence-optical analysis,
a device for carrying out spectrophotometric analysis, and
a device for carrying out enzymatic analysis.

27. The method as claimed in claim 14, wherein the biosensor comprises an arrangement of a plurality of biosensors in order to analyze a plurality of parameters of the part of the tissue volume.

28. The method as claimed in claim 14, wherein a sample from the part of the tissue is guided to the biosensor for analysis by way of a catheter.

29. The method as claimed in claim 28, wherein the biosensor is arranged outside of the five-Gauss region, wherein the five-Gauss region defines a region in which the magnetic field strength is greater than 5 Gauss.

30. The method as claimed in claim 14, wherein particles with antibodies, oligonucleotides, enzymes or other specific binding molecules are supplied to the tissue volume by way of a catheter, and wherein the analysis of the tissue volume is carried out with the aid of the particles.

31. The method as claimed in claim 14, wherein ferrous particles are supplied to the tissue volume by way of a catheter, and wherein, depending on the analysis of the tissue volume, the tissue volume is excited by an alternating magnetic field in order to increase the temperature in the tissue volume.

32. The method as claimed in claim 14, wherein particles containing a medical substance encapsulated in a thermo-sensitive substance are supplied to the tissue volume by way of a catheter and wherein, depending on the analysis of the tissue volume, the tissue volume is heated in order to release the medical substance in the tissue volume in a targeted fashion.

33. The device as claimed in claim 2, wherein a coil is arranged on the biosensor to improve imaging of the biosensor by way of a magnetic resonance scanner.

34. The device as claimed in claim 5, wherein a coil is arranged on the sample acquisition device to improve imaging of the sample acquisition device by way of a magnetic resonance scanner.

35. The device as claimed in claims 5, the container is developed to hold particles with antibodies, oligonucleotides, enzymes or other specific binding molecules, wherein the device is developed such that the pump can be used to pump the particles through the catheter to the distal end of the catheter and wherein the biosensor is developed for real-time analysis by way of the particles.

36. A computer readable medium including program segments for, when executed on a computer device, causing the computer device to implement the method of claim 12.

37. A computer readable medium including program segments for, when executed on a computer device, causing the computer device to implement the method of claim 14.
